(19) United States
(12) Patent Application Publication
(10) Pub. No.: US 2024/0402170 A1
(43) Pub. Date: Dec. 5, 2024

(54) COMBINATION OF HOST RESPONSE AND DIRECT ANTIGEN DETECTION FOR DETECTION OF INFECTION CAUSED BY FASTIDIOUS ORGANISMS

(71) Applicant: CD DIAGNOSTICS, INC., Claymont, DE (US)

(72) Inventors: Krista Toler, Pierceton, IN (US); Carl Deirmengian, Newton Square, PA (US)

(21) Appl. No.: 18/679,174

(22) Filed: May 30, 2024

Related U.S. Application Data
(60) Provisional application No. 63/470,640, filed on Jun. 2, 2023.

(51) Int. Cl. G01N 33/569

(52) U.S. Cl. CPC G01N 33/56911; G01N 2333/195; G01N 2469/10

(57) ABSTRACT

Various aspects disclosed relate to a method for detecting infection, the method includes testing a synovial fluid sample for the presence of a predetermined pathogen. The method further includes testing the synovial fluid sample for a measure of infection. The method further includes determining a rank score based on the results of the testing for the presence of the predetermined pathogen and the results of the testing for a measure of infection. The method further includes determining if the rank score is above a threshold. An infection is deemed present if the rank score is above the threshold and the testing for the presence of a predetermined pathogen does not comprise microbiological culture.

### CROSS-REFERENCE TO RELATED APPLICATION

This application claims the benefit of priority to U.S. Provisional Patent Application Ser. No. 63/470,640 entitled “COMBINATION OF HOST RESPONSE AND DIRECT ANTIGEN DETECTION FOR DETECTION OF INFECTION CAUSED BY FASTIDIOUS ORGANISMS,” filed Jun. 2, 2023, the disclosure of which is incorporated herein in its entirety by reference.

### BACKGROUND

Joint infection is difficult to diagnose, and existing diagnostic criteria use some combination of host response biomarkers with binary cutoff points together with microbiological culture detection of a pathogen. Many times, this combination creates confusion because there is evidence of a host inflammatory response with no pathogen detected or vice versa.

### SUMMARY OF THE INVENTION

Various aspects disclosed relate to a method for detecting infection, the method includes testing a synovial fluid sample for the presence of a predetermined pathogen. The method further includes testing the synovial fluid sample for a measure of infection. The method further includes determining a rank score based on the results of the testing for the presence of the predetermined pathogen and the results of the testing for a measure of infection. The method further includes determining if the rank score is above a threshold. An infection is deemed present if the rank score is above the threshold and the testing for the presence of a predetermined pathogen does not comprise microbiological culture.

Various aspects disclosed relate to a method for detecting infection. The method includes testing a synovial fluid sample obtained from a shoulder for the presence an antigen associated with a predetermined pathogen. The method further includes testing the synovial fluid sample for a measure of infection. The method further includes determining a rank score based on the results of the testing for the presence of the predetermined pathogen and the results of the testing for a measure of infection. The method further includes determining if the rank score is above a threshold. An infection is deemed present if the rank score is above the threshold and the testing for the presence of a predetermined pathogen does not include cultivating a microbiological culture.

Various aspects disclosed relate to a method for detecting infection. The method includes testing a synovial fluid sample for the presence of a predetermined pathogen. The method further includes testing the synovial fluid sample for a measure of infection. The method further includes determining a rank score based on the results of the testing for the presence of the predetermined pathogen and the results of the testing for a measure of infection. The method further includes determining if the rank score is above a threshold. The method further includes treating the infection if the rank score is above the threshold. An infection is deemed present if the rank score is above the threshold and the testing for the presence of a predetermined pathogen does not comprise microbiological culture.

### BRIEF DESCRIPTION OF THE FIGURES

The drawings illustrate generally, by way of example, but not by way of limitation, various aspects of the present invention.

FIG. 1 is a block diagram of an example of an environment including a system for neural network training.

FIG. 2 is a violin plot of the inflammatory rank-percentiles across the entire study demonstrates a bimodal distribution, with one mode centered at the 35^{th} percentile and another mode centered at the 90^{th} percentile. Bold line=population median; standard lines=interquartile ranges.

FIG. 3 is a violin plot of reference and study groups demonstrates their respective population distributions as a function of the inflammatory rank-percentile. Cx=culture; Ag=antigen. The asterisk denotes statistical significance with all p<0.0001 of the elevated median inflammatory rank-percentile compared to both low inflammatory groups. Bold lines are medians whereas normal lines denote interquartile ranges.

FIG. 4 is a bar graph demonstrating the median and interquartile ranges of C. acnes antigen levels of reference and study groups. Cx=culture; Ag=antigen. The asterisk denotes statistical significance with all p<0.001 compared to the three groups with low antigen levels.

FIG. 5 is a scatter plot of all C. acnes culture(+) samples in the study demonstrating two clusters differentiated by antigen levels and inflammatory rank-percentile. Red dots=samples that are C. acnes antigen(+). Black dots=samples that are C. acnes antigen (−). S/CO=signal/cutoff.

FIG. 6 is a violin plot demonstrating the frequency of days needed for positivity of the C. acnes culture. Bold lines are medians whereas normal lines denote interquartile ranges. The asterisk denotes statistical significance (p<0.0001) when comparing the median hours needed to yield C. acnes in culture. Cx=culture; Ag=antigen.

### DETAILED DESCRIPTION OF THE INVENTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. A comma can be used as a delimiter or digit group separator to the left or right of a decimal mark; for example, “0.000,1” is equivalent to “0.0001.” All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

Joint infection, such as native joint infection and periprosthetic joint infection (“PJI”), sepsis, bacterial meningitis, and other forms of microbial (e.g., bacterial, fungal, viral, protozoal) infection can be debilitating and difficult to diagnose. It is important to diagnose microbial infection and determine the identity of the infecting microorganism in order to inform treatment, which can include selection of an appropriate pharmacological intervention for the specific microorganism causing the infection. The classic means or “gold standard” for determining whether a microbial infection is present in a patient tissue or fluid sample, is to culture the sample of tissue and observe the culture for the growth of microorganisms. However, culture methods have long time-to-results (requiring several days to weeks for culture growth), and also have high false negative rates because not all infected samples will be evidenced by culture growth, e.g., only approximately 50% of actual PJI infections are evidenced in sample culture. This inability to directly detect microbes in patient tissue samples has left physicians without means for routine diagnosis of microbial infections, including PJI. Therefore, physicians often must rely on indirect criteria to diagnose microbial infection.

As a solution to the aforementioned problem, the instantly disclosed method demonstrates that a host response biomarker(s) can be combined to create a rank-percent inflammatory score. This score can be used together with direct antigen detection by immunoassay to differentiate infected synovial fluid samples from not infected synovial fluid samples, independent of microbiological culture.

According to various aspects the disclosed method includes testing a synovial fluid sample for the presence of a predetermined pathogen. According to various aspects, the predetermined pathogen can be a bacteria. Non-limiting examples of suitable bacteria include Cutibacterium acnes, Enterococcus faecalis, Enterococcus faecium, Pseudomonas aeruginosa, Staphylococcus aureus, Staphylococcus epidermidis, Staphylococcus lugdunensis, Staphylococcus warneri, Staphylococcus capitis, Staphylococcus caprae, Streptococcus mitis, Streptococcus oralis, Streptococcus agalactiae, Streptococcus anginosus, Streptococcus gordonii, Escherichia coli, Propionibacterium acnes, Proteus mirabilis, Granulicatella adjacens, Acinetobacter baumannii, Abiotrophia defective, Corynebacterium striatum, Corynebacterium minutissimum, Parvimonas micra, Candida parapsilosis, Candida glabrata, Candida tropicalis, Candida albicans, or a mixture thereof.

Amongst those possible bacteria, Cutibacterium acnes is particularly well suited for detection according to the instantly disclosed method. This is because Cutibacterium acnes is naturally present in the synovial fluid around areas of interest such as a shoulder joint. Therefore, if only a microbial culture test were used to associate the presence of Cutibacterium acnes with an infection, there is a distinct risk that a false positive could be generated. Additionally, Cutibacterium acnes is difficult to grow in a microbial culture, so there is a risk for a false positive if no Cutibacterium acnes is grown. These same considerations apply to other pathogens that may have a risk for generating false positives, false negatives, or both. The instantly disclosed methods however, allow for more meaningful detection of a pathogen and association with a possible infection.

The methods for testing for the presence of a predetermined pathogen can include a polymerase chain reaction assay, a next generation sequencing assay, an immunoassay, or a combination thereof. As generally understood, polymerase chain reaction (PCR) is a technique that can make many copies of a specific DNA segment in a test tube. It uses DNA polymerase, an enzyme that synthesizes new DNA strands from single-stranded templates. PCR can amplify DNA sequences quickly and accurately for various applications in molecular biology, forensic analysis, evolutionary biology, and medical diagnostics.

As generally understood, next-generation sequencing (NGS) is a technology for determining the sequence of DNA or RNA to study genetic variation associated with diseases or other biological phenomena. NGS technology has transformed how clinical researchers and scientists think about genetics, as it assesses multiple genes in a single assay. It can sequence an entire or particular genome of interest within a short period.

Without being so limited, the immunoassay can include preparing an immunoassay capture reagent, or an immunoassay detection reagent, or both, configured to specifically bind to the predetermined pathogen. Additionally, the immunoassay can include a capture reagent, a detection reagent, or both, configured to specifically bind a microorganism isolated from a biological sample obtained from a subject having a joint infection. The methods for testing for the presence of a predetermined pathogen do not include growing a microbial culture of the predetermined pathogen.

More specifically, suitable immunoassays are binding assays involving binding between antibodies and antigen. Many types and formats of immunoassays are well-known and all can be used for detection and identification of microbes in biological samples. Examples of immunoassays include enzyme linked immunosorbent assays (“ELISA”), enzyme linked immunospot assay (“ELISPOT”), radioimmunoassays (“RIA”), radioimmune precipitation assays (“RIPA”), immunobead capture assays, Western blotting, dot blotting, gel-shift assays, flow cytometry, protein arrays, antigen arrays, antibody arrays, multiplexed bead arrays, magnetic capture, in vivo imaging, fluorescence resonance energy transfer (“FRET”), fluorescence recovery/localization after photobleaching (“FRAP/FLAP”), a sandwich immunoassay, a competitive immunoassay, an immunoassay using a biosensor, an immunoprecipitation assay, an agglutination assay, a turbidity assay, a nephelometric assay, immunoPCR, Quanterix, Singulex, AlphaLISA, Siscapa, Luminex®, Singulex Brenna® immunoassay, TR-FRET, Meso-scale discovery (MSD), lateral flow immunochromatographic device, automated magnetic particle assay, fluorescent polarization, chemiluminescence, electrochemiluminescence, etc.

In general, immunoassays involve contacting a biological sample suspected of containing a molecule of interest (an analyte, such as the microbial antigens in the prepare patient sample) with an antibody to the analyte, or contacting an antibody to a analyte with a molecule that can be bound by the antibody, as the case may be, under conditions effective to allow the formation of immune complexes. Contacting a biological sample with the antibody to the analyte or with the molecule that can be bound by an antibody to the analyte under conditions for a period of time sufficient to allow the effective formation of immune complexes (primary immune complexes) is generally a matter of contacting the antibody and the sample and incubating the mixture for a period of time long enough for the antibodies to form immune complexes, e.g., to bind to, any antigens present in the sample to which the antibodies can bind. In many forms of immunoassay, the sample-antibody composition can then be washed to remove any non-specifically bound antibodies or unbound proteins, allowing only those antibodies specifically bound in the primary immune complexes to be detected.

Immunoassays can include methods for detecting or quantifying the amount of analyte (such as the disclosed microbial antigens or their antibodies) in a biological sample, which methods generally involve the detection or quantification of immune complexes formed during the binding process. In general, the detection of immune complex formation is well known in the art and can be achieved through the application of numerous approaches. These methods are generally based upon the detection of a label or tag, such as any radioactive, colored, chemiluminescent, fluorescent, biological or enzymatic tag or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorimetric substrates (e.g., horseradish peroxidase). The use of fluorescent dyes is generally preferred in the practice of the methods disclosed herein.

There are two main types of immunoassays, homogeneous and heterogeneous, each of which may be used in the methods of detecting and identifying microbes described herein. In homogeneous immunoassays, both the immunological reaction between an antigen and an antibody and the detection are carried out simultaneously in a homogeneous reaction. Heterogeneous immunoassays include at least one separation step between bound and unbound label, which allows the differentiation of reaction products from unreacted reagents. A variety of immunoassays can be used to detect one or more of the microorganisms disclosed herein.

ELISA and Luminex® bead-based array platforms are examples of sandwich immunoassays that can be used in the methods disclosed herein. A “sandwich” immunoassay, in which the antigen being assayed is held between two antibodies, is a preferred format for the methods disclosed herein. In this method, a solid substrate is first coated with a solid phase antibody. The test sample, containing the antigen (e.g., a diagnostic protein), or a composition containing the antigen, such as a synovial fluid sample from a subject of interest, can then be added and the antigen can be allowed to react with the substrate-bound antibody. Any unbound antigen can be washed away. A known amount of enzyme-labeled detection antibody can then allowed to react with the bound antigen. Any excess, unbound enzyme-linked antibody can be washed away. The substrate specific for the enzyme used in the assay can then be added and the reaction between the substrate and the enzyme produces a color change. The amount of visual color change is a direct measurement of specific enzyme-conjugated bound antibody, and consequently directly proportional to the amount of antigen present in the sample tested.

In many immunoassays, as described elsewhere herein, detection of antigen is made with the use of antigen-specific antibodies as detection molecules. However, immunoassays and the systems and methods of the present invention are not limited to the use of antibodies as detector molecules. Any substance that can bind or capture the antigen within a given sample may be used. Aside from antibodies, suitable substances that can also be used as detector molecules include but are not limited to enzymes, peptides, proteins, receptors, and nucleic acids. Further, there are many detection methods known in the art in which the captured antigen may be detected. In some assays, enzyme-linked antibodies produce a color change. In other assays, detection of the captured antigen is made through detecting fluorescent, luminescent, chemiluminescent, or radioactive signals. The system and methods of the current invention is not limited to the particular types of detectable signals produced in an immunoassay.

The method further includes testing the synovial fluid sample for a measure of infection. The measure of infection comprises determining a host inflammatory state, analyzing a concentration of a metabolite associated with infection, or a combination thereof. Non-limiting examples of metabolites can include a lipid, a cholesterol, an N-acetylated molecule, citrate, glycine, glycosaminoglycans, creatinine, histidine, lysine, formate, glucose, proline, valine, dimethylsulfone, mannose, glutamine, or a mixture thereof. Additionally, testing for the measure of infection can include conducting immunoassays for alpha-defensin (SF-AD), human neutrophil elastase (SF-HNE), or interleukin-8 (SF-IL-8), white blood cell count (SF-WBC) and polymorphonuclear cell percent (SF-PMN %), clinical chemistry tests for synovial fluid C-reactive protein (SF-CRP) or lactate, or any combination thereof.

The results from the testing for the presence of the predetermined pathogen and the results of the testing for the measure of infection are combined to determine a rank score. The rank score is compared against a threshold value. If the rank score is above the threshold then an infection is present. The rank score is determined by assigning a rank-percentile (0-100%) that characterizes a sample's inflammatory severity based on synovial fluid biomarkers. Examples, of those biomarkers include: the synovial fluid C-reactive protein (SF-CRP), alpha-defensin (SF-AD), white blood cell count (SF-WBC), and polymorphonuclear cell percent (SF-PMN %).

To calculate a sample's inflammatory rank percentile, an individual sample is ranked compared to a reference data set for each biomarker. Each sample's rank position was then converted to a percentile, providing a standardized rank score for each sample per biomarker. To calculate the final rank score, the mean rank-score is calculated across all biomarkers for each sample.

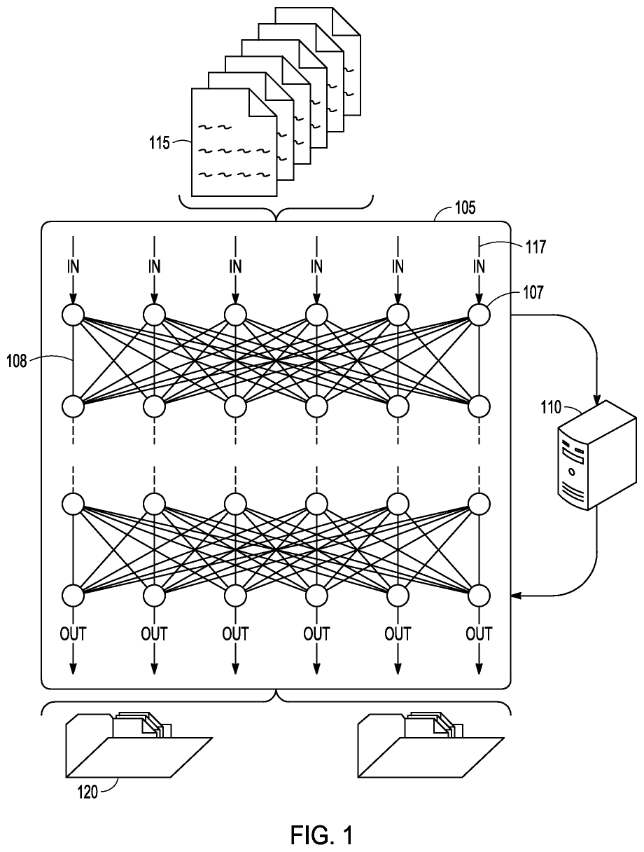

Alternatively or additionally, the results from the testing for the presence of the predetermined pathogen and the results of the testing for the measure of infection can be fed into a machine learning (e.g., neural networking) application to determine the likelihood of infection. FIG. 1 is a block diagram of an example of an environment including a system for neural network training. The analysis operation 128, GNN 104, decoder 330, encoder 430, decoder 426, or the like can include an NN that can be trained in accord with FIG. 1. The resulting NN can predict entity interactions using interacting dynamic graphs. The system includes an artificial NN (ANN) 605 that is trained using a processing node 610. The processing node 610 may be a central processing unit (CPU), graphics processing unit (GPU), field programmable gate array (FPGA), digital signal processor (DSP), application specific integrated circuit (ASIC), or other processing circuitry. In an example, multiple processing nodes may be employed to train different layers of the ANN 605, or even different nodes 607 within layers. Thus, a set of processing nodes 610 is arranged to perform the training of the ANN 605.

The set of processing nodes 610 is arranged to receive a training set 615 for the ANN 605. The ANN 605 comprises a set of nodes 607 arranged in layers (illustrated as rows of nodes 607) and a set of inter-node weights 608 (e.g., parameters) between nodes in the set of nodes. In an example, the training set 615 is a subset of a complete training set. Here, the subset may enable processing nodes with limited storage resources to participate in training the ANN 605.

The training data may include multiple numerical values representative of a domain, such as a word, symbol, other part of speech, or the like. Each value of the training or input 617 to be classified after ANN 605 is trained, is provided to a corresponding node 607 in the first layer or input layer of ANN 605. The values propagate through the layers and are changed by the objective function.

As noted, the set of processing nodes is arranged to train the neural network to create a trained neural network. After the ANN is trained, data input into the ANN will produce valid classifications 620 (e.g., the input data 617 will be assigned into categories), for example. The training performed by the set of processing nodes 607 is iterative. In an example, each iteration of the training the ANN 605 is performed independently between layers of the ANN 605. Thus, two distinct layers may be processed in parallel by different members of the set of processing nodes. In an example, different layers of the ANN 605 are trained on different hardware. The members of different members of the set of processing nodes may be located in different packages, housings, computers, cloud-based resources, etc. In an example, each iteration of the training is performed independently between nodes in the set of nodes. This example is an additional parallelization whereby individual nodes 607 (e.g., neurons) are trained independently. In an example, the nodes are trained on different hardware.

Importantly, the same sample of synovial fluid is used to both test for the presence of the predetermined pathogen and to testing for a measure of infection. Two discrete synovial fluid samples are not required.

If an infection is present, the infection can be any type of infection produced from a pathogen. As a particular example, the infection can be a septic arthritis or periprosthetic joint infection. The synovial fluid can be taken from various locations, however, the disclosed method is particularly well suited to test for the presence of an infection in synovial fluid taken from a shoulder joint. The synovial fluid can be taken from a human subject, but the disclosed method has veterinary applications such that the synovial fluid can be taken from any animal.

If an infection is detected, the method can further include the step of treating the infection. Treating the infection can include administering an antimicrobial agent. With respect to septic arthritis and periprosthetic joint infection, Treatment will depend on a patient's symptoms, age, and general health. It will also depend on how severe the condition is. Septic arthritis often needs treatment right away with antibiotics. This can improve symptoms within 48 hours. Some infections caused by fungi need treatment with antifungal medicine. Viral infections are not treated with medicine. A fluid called pus may be drained from the joint. A buildup of pus can damage the joint. The pus is drained with a needle, tube, or surgery. It is possible that pus may need to be drained multiple times from the joint over the course of treatment. Other treatment may include: antibiotics, medicines for pain and fever, physical therapy to keep muscle strength, and/or a splint on the joint to relieve pain

### Examples

Various aspects of the present invention can be better understood by reference to the following Examples which are offered by way of illustration. The present invention is not limited to the Examples given herein.

### Methods

### Participants/Samples

All synovial fluid samples considered for inclusion in this study were sent to the clinical laboratory for the purpose of clinical diagnostic testing, potentially including testing for synovial fluid C-reactive protein (CRP), alpha-defensin, synovial fluid white blood cell count (SF-WBC), synovial fluid polymorphonuclear cell count (SF-PMN %), synovial fluid culture, and C. acnes antigen testing. None of the samples were collected specifically for the purpose of this Example, as all were submitted to the clinical laboratory for diagnostic testing. The criteria for sample inclusion in this Example consist of 1) synovial fluid from shoulder, 2) performance of C-acnes antigen testing, 3) performance of microbiologic culture of synovial fluid.

### Demographics, Study Population

Synovial fluid samples utilized for this study were received from 363 distinct clinical sites across the United States. Given the deidentified nature of the sample result database, clinical and demographic information beyond synovial fluid results and location of aspiration (office vs. operating room) were not available for this study. 1,414 synovial fluid samples were identified to be from the shoulder, and also had microbiological cultures and C. acnes immunoassay testing. Of these, 1,126 were from a shoulder arthroplasty and 288 were from a native shoulder.

### Laboratory Tests

All laboratory testing in this study was performed in one clinical laboratory, certified by the Clinical Laboratory Improvement Amendments (CLIA) for testing in general immunology, bacteriology, hematology, and routine chemistry. The SF-WBC and SF-PMN % were performed using automated laboratory cell counting instruments, and all SF-WBC counts >3000 cells/ul were also counted manually to confirm the result as previously described. The synovial fluid CRP and alpha-defensin were all performed by validated immunoassays. Synovial fluid culture was performed using the BacT/Alert (Biomerieux, Inc; https://www.biomerieux-usa.com) facultative aerobic and anaerobic bottles, and the BacT/Alert system to monitor for growth. For shoulder samples, laked (defibrinated) horse blood was supplemented to the anaerobic culture bottle. All shoulder sample cultures in the laboratory are time-extended to a total of two weeks to allow sufficient time for C. acnes growth.

The C. acnes antigen immunoassay test used in this study (CD Laboratories, Zimmer Biomet, Towson MD) was designed to directly detect C. acnes antigen in synovial fluid. It is a laboratory-based immunoassay with a reportable result available within 12 hours of laboratory receipt of the synovial fluid sample. The assay threshold for synovial fluid positivity was determined and set before the samples included in this study were processed by the laboratory and was normalized to a signal/cutoff (S/CO) of 1.0. Therefore, the results of this Example are based on a predetermined assay positivity threshold. Results of the C. acnes assay can be reported as negative, positive, or indeterminate. Only 1 sample included in this Example was clinically reported as indeterminate and was regarded as negative for analysis in this study. Raw numerical signal/cutoff (S/CO) results were available for comparison between groups.

### Inflammatory Rank-Percentile

All synovial fluid samples in this Example were ranked and assigned a rank-percentile (0-100%) characterizing the sample's inflammatory severity based on synovial fluid biomarkers: the synovial fluid C-reactive protein (SF-CRP), alpha-defensin (SF-AD), white blood cell count (SF-WBC), and polymorphonuclear cell percent (SF-PMN %). Of the 1,414 samples included, 71.8% had 4 tests, 22.1% had 3 tests, 5.0% had 2 tests, 1.1% had 1 test completed, with 98.9% of all samples having 2 or more synovial fluid tests available to calculate inflammatory severity. Of the 85 samples that had cultures positive for C. acnes, 95.3% had 3 or more biomarkers available for inflammatory rank-percentile scoring. Correlation matrix analysis of the SF-CRP, SF-AD, SF-WBC, and SF PMN % in our institutional database revealed a minimum correlation (Spearman r) of 0.33 (PMN-CRP) and a maximum correlation of 0.59 (AD-PMN) between individual biomarkers, demonstrating no predominantly redundant biomarker. Therefore, all markers were equally weighted.

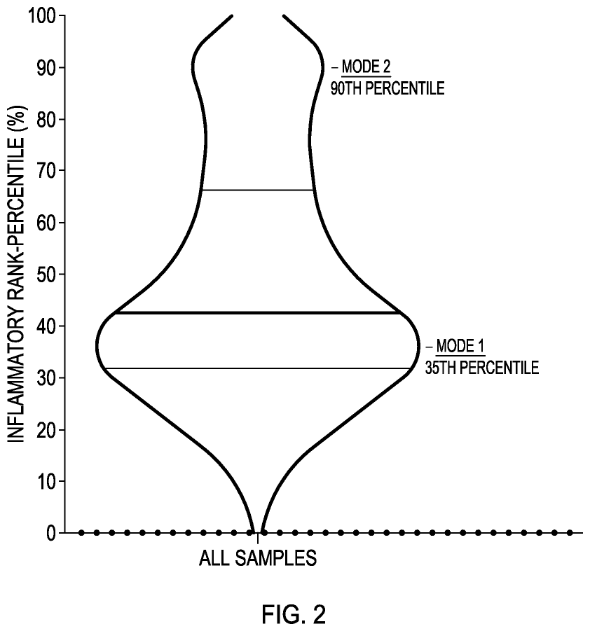

To calculate each sample's inflammatory rank percentile, all samples in this study were ranked from 1 to 1,114 for each biomarker. Each sample's rank position was then converted to a percentile, providing a standardized rank-percentile for each sample per biomarker. To calculate the final inflammatory rank-percentile, the mean rank-percentile was calculated across all biomarkers for each sample in this study. For example, one sample in this study ranked in the 85^{th}, 97^{th}, 99^{th}, and 92^{nd} percentile among all samples for the SF-CRP, SF-AD, SF-WBC, and SF-PMN % respectively. This sample's inflammatory rank-percentile was therefore 93.3% (mean of all biomarker percentiles) among all study samples. The mean laboratory values were determined for a series of rank-percentile ranges to provide a reference across the study (Table 1). Additionally, the distribution of inflammatory rank-percentiles was plotted to demonstrate the entire study population (N=1,414) (FIG. 2).

TABLE 1
Reference table demonstrating relationship between inflammatory rank-percentile ranges
and associated mean laboratory values. SF-CRP = synovial fluid C-reactive protein;
SF-WBC = synovial fluid white blood cells; SF-PMN = synovial fluid polymorphonuclear
cell. S/CO = signal/cutoff.

| | | SF-AD | | | Culture |
|---|---|---|---|---|---|
| | SF-CRP | mean | SF-WBC | SF-PMN % | Positivity |
| Inflammatory | mean | S/CO | mean, cells/μl | mean, % | mean, % |
| Rank-percentile | (95% CI) | (95% CI) | (95% CI) | (95% CI) | (95% CI) |
| 0-5% | — | 0 | 2 | — | — |
| | | (0-0.1) | (0-4) | | |
| 5-15% | 0 | 0.1 | 72 | 23% | 9.7% |
| | (0-0.1) | (0.1-0.1) | (39-106) | (18-27) | (0.9-20.3) |
| 15-25% | 0.1 | 0.1 | 257 | 28% | 5.0% |
| | (0.1-0.2) | (0.1-0.1) | (205-310) | (25-31) | (1.4-8.6) |
| 25-35% | 0.7 | 0.1 | 470 | 36% | 4.9% |
| | (0.5-0.9) | (0.1-0.1) | (404-537) | (33-38) | (2.4-7.4) |
| 35-45% | 1.2 | 0.1 | 741 | 36% | 4.9% |
| | (1-1.4) | (0.1-0.1) | (658-825) | (34-39) | (2.5-7.4) |
| 45-55% | 2.2 | 0.1 | 1,044 | 43% | 2.4% |
| | (1.6-2.8) | (0.1-0.2) | (893-1,195) | (40-45) | (0.1-4.7) |
| 55-65% | 3.2 | 0.3 | 1,663 | 50% | 8.0% |
| | (2.4-4) | (0.2-0.3) | (1,397-1,929) | (47-53) | (3.0-13.1) |
| 65-75% | 6.5 | 0.5 | 4,248 | 64% | 7.9% |
| | (4-9.1) | (0.4-0.6) | (2,378-6,118) | (61-68) | (2.6-13.2) |
| 75-85% | 9.7 | 1.0 | 6,247 | 78% | 25.6% |
| | (6.4-12.9) | (0.9-1.2) | (5,191-7,302) | (75-81) | (16.3-34.9) |
| 85-95% | 21.1 | 2.7 | 60,883 | 92% | 70.7% |
| | (17.4-24.8) | (2.5-2.9) | (49,406-72,360) | (91-93) | (63.6-77.8) |
| 95-100% | 57.4 | 3.5 | 155,671 | 96% | 79.2% |
| | (44.4-70.4) | (3.3-3.8) | (53,815-257,526) | (95-97) | (62.6-95.8) |

### Reference Groups

Two reference groups were created to provide reference comparisons for the study groups. The culture-negative reference group (N=1,180) included all samples in the study that were culture-negative and C. acnes antigen-negative, which presumably consists substantially of non-infected samples. The culture-positive reference group (N=127) included all culture-positive samples, except those that yielded C. acnes. This culture-positive reference presumably consists substantially of infected samples.

### Study Groups

Two study groups were defined relative to their C. acnes culture and antigen results. One group included samples that are C. acnes culture(+)/antigen(+). The second included samples that were C. acnes culture(+)/antigen (−).

A third study group included samples that yielded no organisms on culture but had a positive C. acnes antigen test.

### Statistical Analysis

The variables utilized for data analysis included C. acnes synovial fluid culture results (binary), C. acnes antigen test results (binary or continuous), and inflammatory rank-percentile (continuous). The Mann-Whitney non-parametric test for significance was used to determine statistical significance between two medians. The Kruskal-Wallis non-parametric test for significance, with Dunn's multiple comparisons test, was used to compare median differences between multiple groups. Non-parametric Spearman correlation was used to compare the correlation between multiple biomarkers.

### Results

### Sample Characteristics

The 1,414 shoulder synovial fluid samples in this Example included 1,202 culture-negative and 212 culture-positive synovial fluid samples from the shoulder. Analysis of the inflammatory rank-percentile across all samples revealed a bimodal distribution, with rank-percentile modes at 35% and 90% (FIG. 2). Among the 212 culture-positive shoulder fluid samples, 85 (40.1%) yielded C. acnes and 127 yielded other organisms (Table 2).

TABLE 2
Microorganisms isolated from culture-positive samples in this study.

| Microorganism | N |
|---|---|
| Cutibacterium acnes | 85 |
| Staphylococcus aureus | 32 |
| Staphylococcus | |
| epidermidis | 30 |
| Serratia marcescens | 5 |
| Streptococcus mitis/oralis | 3 |
| Parvimonas micra | 3 |
| Streptococcus agalactiae | 3 |
| Staphylococcus | |
| lugdunensis | 2 |
| Staphylococcus | |
| haemolyticus | 2 |
| Pseudomonas aeruginosa | 2 |
| Streptococcus anginosus | 2 |
| Streptococcus mutans | 2 |
| Enterococcus faecalis | 2 |
| Streptococcus sanguinis | 2 |
| Staphylococcus capitis | 2 |
| Proteus mirabilis | 2 |
| Other | 33 |
| Total | 212 |

### Reference Groups

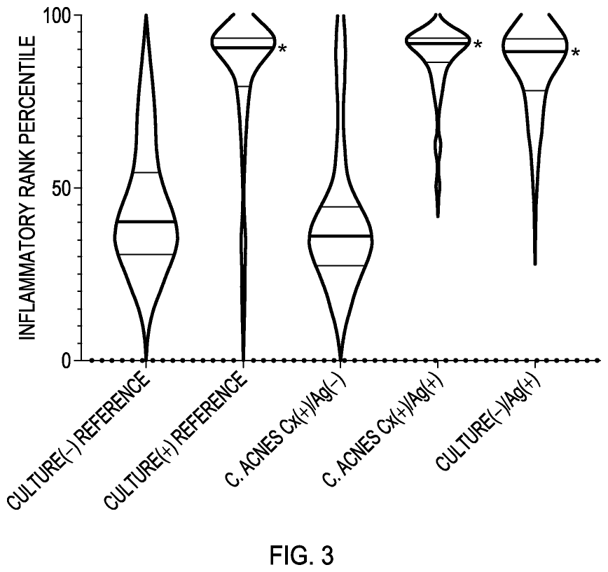

Inflammatory rank-percentiles were calculated for two reference populations:
culture-negative samples (excluding those with a positive C. acnes antigen test) and culture-positive samples (excluding those that were C. acnes culture-positive) (FIG. 3. Table 3).

TABLE 3
Results of C. acnes antigen testing, inflammatory rank-percentile,
and time to culture growth for reference samples and study samples.

| | | | | | | Time to |
|---|---|---|---|---|---|---|
| | | | | C. acnes | Inflammatory | culture |
| | | C. acnes | C. acnes | Antigen | Rank-Percentile | C. acnes |
| | | culture | antigen | (median S/CO) | Median % | Median days |
| Group | N | result | test | (95% CI) | (95% CI) | (95% CI) |
| Reference | | | | | | |
| Groups | | | | | | |
| Culture(−) | 1,180 | — | — | 0.1 | 40.2% | — |
| reference | | | | (0.1-0.1) | (38.9-41.2%) | |
| Culture(+) | 127 | — | — | 0.1 | 90.4% | — |
| reference | | | | (0.1-0.1) | (88.5-91.9%) | |
| Study Groups | | | | | | |
| Cluster 1 - C. acnes | 34 | Positive | Negative | 0.1 | 36.0% | 9.7 |
| Culture(+)/antigen(−) | | | | (0.1-0.2) | (30.5-41.8%) | (8.0-11.6) |
| Cluster 2 - C. acnes | 51 | Positive | Positive | 16.6 | 91.7% | 6.7 |
| Culture(+)/antigen(+) | | | | (11.7-22.6) | (89.2-92.5%) | (5.5-7.8) |
| C. acnes | 22 | Negative | Positive | 8.8 | 89.4% | — |
| Culture(−)/antigen(+) | | | | (2.6-17.4) | (79.3-93.0%) | |

S/CO = signal/cutoff.

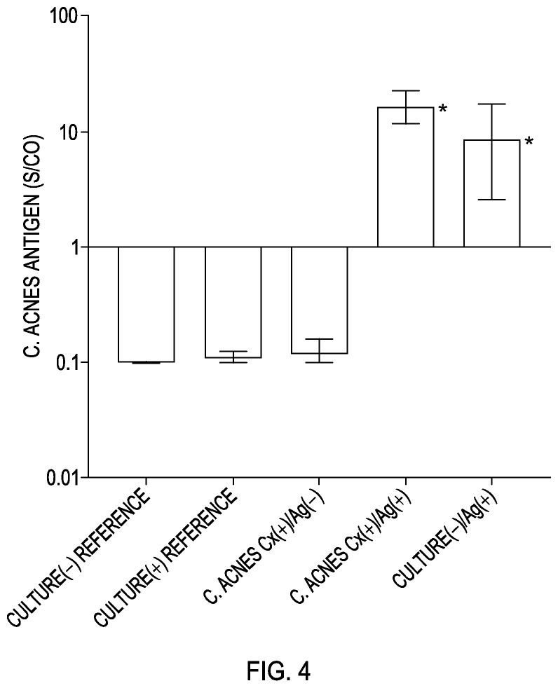

Culture-negative reference samples (N=1,180) had a median inflammatory rank-percentile of 40.2% (95% CI: 38.9 to 41.2%) and median C. acnes antigen level of 0.1 (S/CO) (95% CI: 0.1 to 0.1 [S/CO]) (FIG. 4).

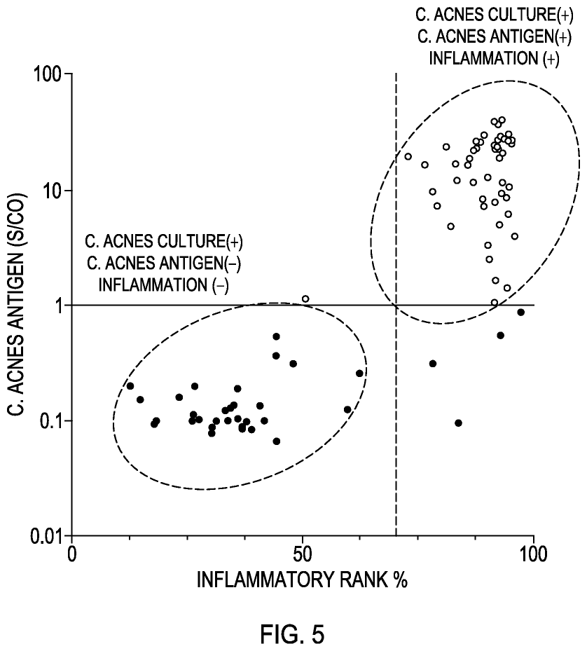

Culture-positive reference samples (N=127) had a median inflammatory rank-percentile of 90.4% (95% CI: 88.5 to 91.9%) and median C. acnes antigen level of 0.1 (S/CO) (95% CI: 0.1 to 0.1 [S/CO]) (FIG. 5).

### Study Groups

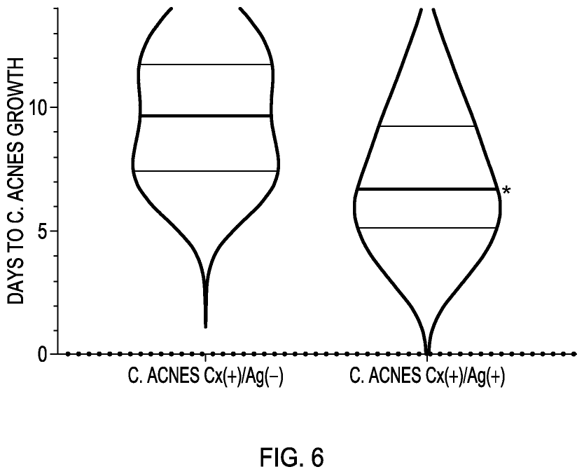

Two distinct clusters (FIG. 5) of C. acnes culture-positive samples were identified, which could be differentiated by inflammatory rank-percentile (Table 3, FIG. 3), C. antigen levels (FIG. 4), and hours to C. acnes culture growth (FIG. 6).

Cluster 1 (N=34) consisted of samples that were C. acnes culture(+)/antigen (−), demonstrating a low median inflammatory rank-percentile of 36.0%, a low median antigen level of 0.1 (S/CO), and a delayed median time to culture growth of 9.7 days. Only 11.7% (4/34) of these samples had an inflammatory rank-percentile over 75%, and 20.6% (7/34) had culture growth within 7 days. This group demonstrated a median inflammatory rank-percentile that was most similar to the culture-negative reference group (36.0 vs. 40.2%; p>0.99) (Table 3).

Cluster 2 (N=51) consisted of samples that were C. acnes culture(+)/antigen(+), demonstrating a high median inflammatory rank-percentile of 91.7%, a high median antigen level of 16.6 (S/CO), and an early median time to culture growth of 6.7 days. Of these samples, 92.2% (47/51) had an inflammatory rank-percentile over 75%, and 52.9% (27/51) had culture growth within 7 days. The increased inflammatory rank-percentile, increased C. acnes antigen levels, and lower hours to culture growth in this cluster were all different than that of cluster 1 with statistical significance (all p<0.0001). This group demonstrated a median inflammatory rank-percentile that was most similar to the culture-positive reference group (91.7 vs. 90.4%; p>0.99) (Table 3).

Among all culture-negative samples in this study were 22 samples that were C. acnes antigen(+), which demonstrated a median inflammatory rank-percentile of 89.4%, and a median antigen level of 8.8 (S/CO) (Table 3, FIG. 4). The median inflammatory rank-percentile in this group (FIG. 3) was greater that the median among culture-negative reference samples (89.4 vs. 40.2%; p<0.0001) and similar to the median among the culture-positive reference (89.4 vs. 90.4%; p>0.99).

### Discussion

This Example has demonstrated the stratification of C. acnes culture-positive synovial fluid samples into two distinct clusters using C. acnes antigen testing yielding 1) a cluster characterized by absence of C. acnes antigen, low inflammation, and a longer time to culture, versus 2) a cluster characterized by C. acnes antigen positivity, high inflammation, and a shorter time to culture. The antigen-positive inflammatory cluster of C. acnes culture-positive samples was further demonstrated to have inflammation levels on par with reference samples that were culture-positive for organisms other that C. acnes, suggesting the presence of infection. The antigen-negative non-inflammatory cluster of C. acnes culture-positive samples was demonstrated to have inflammation levels similar to a large reference cohort of culture-negative samples in the study, suggesting the absence of infection. Additionally, a small subset of culture-negative samples which tested positive for C. acnes antigen demonstrated substantial inflammation on par with culture-positive reference samples, indicating the detection of culture-negative C. acnes infection by the antigen test.

The existing C. acnes literature has established a paradigm which suggests that C. acnes culture-positive samples may be separated into those that indicate true clinical infection, demonstrating a host inflammatory response and early time to culture, versus those due to contamination characterized by the absence of host response and delayed culture growth. For example, a previously described shorter median time to culture positivity (median; 5 days vs. 9 days) among samples from patients with probable true-positive infection versus those with probable contamination. Others have described the time to culture positivity at several laboratories when comparing growth from a sample that was inoculated with C. acnes versus growth from negative-control samples that were sterile. They observed that C. acnes growth from samples inoculated with C. acnes had a mean time to growth of 4 days, compared to a mean of 8.3 required days to culture-positivity for sterile control samples. Additionally, several institutions have suggested that C. acnes culture-positivity, in the absence of clinical or laboratory evidence of an inflammatory host response, may represent contamination and might be safely treated without special consideration. Others have found that patients with an unexpected positive culture at the time of primary arthroplasty did not have inferior clinical outcomes or higher infection rates post operatively.

The instant disclosure confirms this existing paradigm by demonstrating the presence of two distinct populations in a large laboratory sample population, distinguished by their inflammatory host response profile and time to culture growth. However, this study goes further in demonstrating that the samples with a substantial inflammatory host response and early median culture growth are predominantly C. acnes antigen-positive, while those samples with no inflammatory host response and delayed median culture growth are predominantly negative for C. acnes antigen. These new findings not only build upon the existing paradigm of true-positive versus false-positive C. acnes culture, but also provide a useful diagnostic tool to aid in the differentiation of these two distinct populations.

The association of positive C. acnes antigen levels with an elevated host inflammatory response and early time to culture is most easily explained by a hypothesis based on the C. acnes bacterial load present in the synovial fluid when received by the laboratory. Synovial fluid samples with higher C. acnes loads will 1) have positive antigen testing, 2) be expected to induce a higher level of host inflammatory response, and 3) require fewer doublings in culture to achieve culture growth. Clinical infection by C. acnes would be the most obvious mechanism leading to a sufficient bacterial load to trigger positive antigen testing, a host inflammatory response, and early culture growth. To the contrary, contamination would likely present log-fold lower C. acnes bacterial loads, leading to negative antigen testing, absence of an inflammatory host response, and late culture growth.

An additional finding in this Example was that 22 samples demonstrated positive C. acnes antigen testing despite having a negative culture result. The median inflammatory rank-percentile in this group matched that of C. acnes culture(+)/antigen(+) samples (89.4% vs. 91.7%, p>0.99), and also matched the inflammatory characteristics of the culture-positive reference group in this study (89.4% vs. 90.4%, p>0.99). All 22 culture-negative antigen-positive samples had inflammation that ranked in the top 50^{th} percentile in this study, and 17 of these samples ranked above the 75^{th} inflammatory percentile in this study. These findings strongly suggest that these 22 samples with positive antigen levels reflect culture-negative C. acnes infections.

Although the large size of samples and breadth of locations sending samples may have minimized institutional aspiration biases, it also prevented the establishment of clinical inclusion and exclusion criteria to produce a well-defined population. This consideration, combined with the lack of a clinically-accepted gold-standard to diagnose C. acnes shoulder infection, resulted in the inability to define the diagnostic performance of the C. acnes antigen test, but instead describe its association with inflammation and culture results. Nevertheless, the association of antigen-positivity with the host inflammatory response and time to culture growth contributes a new and considerably important finding to our understanding of C. acnes shoulder infection.

In conclusion, this Example has provided a first report of C. acnes antigen testing for synovial fluid from the shoulder, demonstrating the potential stratification of true-positive cultures from contamination. Furthermore, the study demonstrates the potential for antigen testing to provide a novel method of detecting culture-negative C. acnes infection.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### EXEMPLARY ASPECTS

The following exemplary aspects are provided, the numbering of which is not to be construed as designating levels of importance:

Aspect 1 provides a method for detecting infection, the method comprising:
testing a synovial fluid sample for the presence of a predetermined pathogen;
testing the synovial fluid sample for a measure of infection;
determining a rank score based on the results of the testing for the presence of the predetermined pathogen and the results of the testing for a measure of infection; and
determining if the rank score is above a threshold, wherein
an infection is present if the rank score is above the threshold and the testing for the presence of a predetermined pathogen does not comprise microbiological culture.

Aspect 2 provides the method of Aspect 1, wherein the testing for the presence of a predetermined pathogen comprises a polymerase chain reaction assay, a next generation sequencing assay, an immunoassay, or a combination thereof.

Aspect 3 provides the method of Aspect 2, wherein performing the immunoassay comprises preparing an immunoassay capture reagent, or an immunoassay detection reagent, or both, configured to specifically bind to the predetermined pathogen.

Aspect 4 provides the method of Aspect 3, wherein the immunoassay comprises a capture reagent, a detection reagent, or both, configured to specifically bind a microorganism isolated from a biological sample obtained from a subject having a joint infection.

Aspect 5 provides the method of any of Aspects 1-4, wherein the measure of infection comprises determining a host inflammatory state, analyzing a concentration of a metabolite associated with infection, or a combination thereof.

Aspect 6 provides the method of Aspect 5, wherein the metabolite comprises a lipids, a cholesterol, an N-acetylated molecule, citrate, glycine, glycosaminoglycans, creatinine, histidine, lysine, formate, glucose, proline, valine, dimethylsulfone, mannose, glutamine, or a mixture thereof.

Aspect 7 provides the method of Aspect 5, wherein the measure of infection comprises immunoassays for alpha-defensin (SF-AD), human neutrophil elastase (SF-HNE), or interleukin-8 (SF-IL-8), white blood cell count (SF-WBC) and polymorphonuclear cell percent (SF-PMN %), clinical chemistry tests for synovial fluid C-reactive protein (SF-CRP) or lactate, or any combination thereof.

Aspect 8 provides the method of any of Aspects 1-7, further comprising treating the infection.

Aspect 9 provides the method of Aspect 8, wherein treating the infection comprises administering an antimicrobial agent.

Aspect 10 provides the method of any of Aspects 1-9, wherein the infection is a septic arthritis or periprosthetic joint infection.

Aspect 11 provides the method of any of Aspects 1-10, wherein the synovial fluid is obtained from a shoulder joint.

Aspect 12 provides the method of any of Aspects 1-11, wherein the predetermined pathogen is Cutibacterium acnes, Enterococcus faecalis, Enterococcus faecium, Pseudomonas aeruginosa, Staphylococcus aureus, Staphylococcus epidermidis, Staphylococcus lugdunensis, Staphylococcus warneri, Staphylococcus capitis, Staphylococcus caprae, Streptococcus mitis, Streptococcus oralis, Streptococcus agalactiae, Streptococcus anginosus, Streptococcus gordonii, Escherichia coli, Propionibacterium acnes, Proteus mirabilis, Granulicatella adjacens, Acinetobacter baumannii, Abiotrophia defective, Corynebacterium striatum, Corynebacterium minutissimum, Parvimonas micra, Candida parapsilosis, Candida glabrata, Candida tropicalis, Candida albicans, or a mixture thereof.

Aspect 13 provides the method of any of Aspects 1-12, wherein the predetermined bacteria is Cutibacterium acnes.

Aspect 14 provides the method of any of Aspects 1-13, wherein testing for the presence of a predetermined pathogen and testing for a measure of infection to determine a rank score are accomplished using the same synovial fluid sample.

Aspect 15 provides the method of any of Aspects 1-14, wherein the synovial fluid is obtained from a human.

Aspect 16 provides the method of any of Aspects 1-14, wherein the synovial fluid is obtained from an animal.

Aspect 17 provides a method for detecting infection, the method comprising:
testing a synovial fluid sample obtained from a shoulder for the presence an antigen associated with a predetermined pathogen;
testing the synovial fluid sample for a measure of infection;
determining a rank score based on the results of the testing for the presence of the predetermined pathogen and the results of the testing for a measure of infection; and
determining if the rank score is above a threshold, wherein
an infection is present if the rank score is above the threshold and the testing for the presence of a predetermined pathogen does not comprise microbiological culture.

Aspect 18 provides a method for detecting infection, the method comprising:
testing a synovial fluid sample for the presence of a predetermined pathogen;
testing the synovial fluid sample for a measure of infection;
determining a rank score based on the results of the testing for the presence of the predetermined pathogen and the results of the testing for a measure of infection;
determining if the rank score is above a threshold;
and treating the infection if the rank score is above the threshold, wherein
an infection is present if the rank score is above the threshold and the testing for the presence of a predetermined pathogen does not comprise microbiological culture.

Aspect 19 provides a method for detecting infection, the method comprising:
testing a synovial fluid sample for the presence of a predetermined pathogen;
testing the synovial fluid sample for a measure of infection; and
determining a likelihood of infection based on the results of the testing for the presence of the predetermined pathogen and the results of the testing for a measure of infection.

Aspect 20 provides the method of Aspect 19, wherein the testing for the presence of a predetermined pathogen comprises a polymerase chain reaction assay, a next generation sequencing assay, an immunoassay, or a combination thereof.

Aspect 21 provides the method of Aspect 20, wherein performing the immunoassay comprises preparing an immunoassay capture reagent, or an immunoassay detection reagent, or both, configured to specifically bind to the predetermined pathogen.

Aspect 22 provides the method of Aspect 21, wherein the immunoassay comprises a capture reagent, a detection reagent, or both, configured to specifically bind a microorganism isolated from a biological sample obtained from a subject having a joint infection.

Aspect 23 provides the method of any of Aspects 19-22, wherein the measure of infection comprises determining a host inflammatory state, analyzing a concentration of a metabolite associated with infection, or a combination thereof.

Aspect 24 provides the method of Aspect 23, wherein the metabolite comprises a lipids, a cholesterol, an N-acetylated molecule, citrate, glycine, glycosaminoglycans, creatinine, histidine, lysine, formate, glucose, proline, valine, dimethylsulfone, mannose, glutamine, or a mixture thereof.

Aspect 25 provides the method of Aspect 24, wherein the measure of infection comprises immunoassays for alpha-defensin (SF-AD), human neutrophil elastase (SF-HNE), or interleukin-8 (SF-IL-8), white blood cell count (SF-WBC) and polymorphonuclear cell percent (SF-PMN %), clinical chemistry tests for synovial fluid C-reactive protein (SF-CRP) or lactate, or any combination thereof.

Aspect 26 provides the method of any of Aspects 19-25, further comprising treating the infection.

Aspect 27 provides the method of Aspect 26, wherein treating the infection comprises administering an antimicrobial agent.

Aspect 28 provides the method of any of Aspects 19-27, wherein the infection is a septic arthritis or periprosthetic joint infection.

Aspect 29 provides the method of any of Aspects 19-28, wherein the synovial fluid is obtained from a shoulder joint.

Aspect 30 provides the method of any of Aspects 19-29, wherein the predetermined pathogen is Cutibacterium acnes, Enterococcus faecalis, Enterococcus faecium, Pseudomonas aeruginosa, Staphylococcus aureus, Staphylococcus epidermidis, Staphylococcus lugdunensis, Staphylococcus warneri, Staphylococcus capitis, Staphylococcus caprae, Streptococcus mitis, Streptococcus oralis, Streptococcus agalactiae, Streptococcus anginosus, Streptococcus gordonii, Escherichia coli, Propionibacterium acnes, Proteus mirabilis, Granulicatella adjacens, Acinetobacter baumannii, Abiotrophia defective, Corynebacterium striatum, Corynebacterium minutissimum, Parvimonas micra, Candida parapsilosis, Candida glabrata, Candida tropicalis, Candida albicans, or a mixture thereof.

Aspect 31 provides the method of any of Aspects 19-30, wherein the predetermined bacteria is Cutibacterium acnes.

Aspect 32 provides the method of any of Aspects 19-31, wherein testing for the presence of a predetermined pathogen and testing for a measure of infection are accomplished using the same synovial fluid sample.

Aspect 33 provides the method of any of Aspects 19-32, wherein the synovial fluid is obtained from a human.

Aspect 34 provides the method of any of Aspects 19-33, wherein the synovial fluid is obtained from an animal.

Aspect 35 provides the method of any of Aspects 19-34, wherein determining a likelihood of infection is accomplished using a machine learning algorithm.

What is claimed is:

1. A method for detecting infection, the method comprising:
testing a synovial fluid sample for the presence of a predetermined pathogen;
testing the synovial fluid sample for a measure of infection;
determining a rank score based on the results of the testing for the presence of the predetermined pathogen and the results of the testing for a measure of infection; and
determining if the rank score is above a threshold, wherein
an infection is present if the rank score is above the threshold and the testing for the presence of a predetermined pathogen does not comprise microbiological culture.

2. The method of claim 1, wherein the testing for the presence of a predetermined pathogen comprises a polymerase chain reaction assay, a next generation sequencing assay, an immunoassay, or a combination thereof.

3. The method of claim 2, wherein performing the immunoassay comprises preparing an immunoassay capture reagent, or an immunoassay detection reagent, or both, configured to specifically bind to the predetermined pathogen.

4. The method of claim 3, wherein the immunoassay comprises a capture reagent, a detection reagent, or both, configured to specifically bind a microorganism isolated from a biological sample obtained from a subject having a joint infection.

5. The method of claim 1, wherein the measure of infection comprises determining a host inflammatory state, analyzing a concentration of a metabolite associated with infection, or a combination thereof.

6. The method of claim 5, wherein the metabolite comprises a lipids, a cholesterol, an N-acetylated molecule, citrate, glycine, glycosaminoglycans, creatinine, histidine, lysine, formate, glucose, proline, valine, dimethylsulfone, mannose, glutamine, or a mixture thereof.

7. The method of claim 5, wherein the measure of infection comprises immunoassays for alpha-defensin (SF-AD), human neutrophil elastase (SF-HNE), or interleukin-8 (SF-IL-8), white blood cell count (SF-WBC) and polymorphonuclear cell percent (SF-PMN %), clinical chemistry tests for synovial fluid C-reactive protein (SF-CRP) or lactate, or any combination thereof.

8. The method of claim 1, further comprising treating the infection.

9. The method of claim 8, wherein treating the infection comprises administering an antimicrobial agent.

10. The method of claim 1, wherein the infection is a septic arthritis or periprosthetic joint infection.

11. The method of claim 1, wherein the synovial fluid is obtained from a shoulder joint.

12. The method of claim 1, wherein the predetermined pathogen is Cutibacterium acnes, Enterococcus faecalis, Enterococcus faecium, Pseudomonas aeruginosa, Staphylococcus aureus, Staphylococcus epidermidis, Staphylococcus lugdunensis, Staphylococcus warneri, Staphylococcus capitis, Staphylococcus caprae, Streptococcus mitis, Streptococcus oralis, Streptococcus agalactiae, Streptococcus anginosus, Streptococcus gordonii, Escherichia coli, Propionibacterium acnes, Proteus mirabilis, Granulicatella adjacens, Acinetobacter baumannii, Abiotrophia defective, Corynebacterium striatum, Corynebacterium minutissimum, Parvimonas micra, Candida parapsilosis, Candida glabrata, Candida tropicalis, Candida albicans, or a mixture thereof.

13. The method of claim 1, wherein the predetermined bacteria is Cutibacterium acnes.

14. The method of claim 1, wherein testing for the presence of a predetermined pathogen and testing for a measure of infection to determine a rank score are accomplished using the same synovial fluid sample.

15. The method of claim 1, wherein the synovial fluid is obtained from a human.

16. The method of claim 1, wherein the synovial fluid is obtained from an animal.

17. A method for detecting infection, the method comprising:
testing a synovial fluid sample for the presence of a predetermined pathogen;
testing the synovial fluid sample for a measure of infection;
determining a rank score based on the results of the testing for the presence of the predetermined pathogen and the results of the testing for a measure of infection;
determining if the rank score is above a threshold;
and treating the infection if the rank score is above the threshold, wherein
an infection is present if the rank score is above the threshold and the testing for the presence of a predetermined pathogen does not comprise microbiological culture.

18. A method for detecting infection, the method comprising:
testing a synovial fluid sample for the presence of a predetermined pathogen;
testing the synovial fluid sample for a measure of infection; and
determining a likelihood of infection based on the results of the testing for the presence of the predetermined pathogen and the results of the testing for a measure of infection.

19. The method of claim 18, wherein the testing for the presence of a predetermined pathogen comprises a polymerase chain reaction assay, a next generation sequencing assay, an immunoassay, or a combination thereof.

20. The method of claim 18, wherein the synovial fluid is obtained from a shoulder joint.
